(19) United States
(12) Patent Application Publication
(10) Pub. No.: US 2017/0156812 A1
(43) Pub. Date: Jun. 8, 2017

(54) THERAPEUTIC SKIN LIFTING DEVICE AND RELATED SYSTEMS AND METHODS

(71) Applicant: CARPAL AID, LLC, Chatsworth, CA (US)

(72) Inventor: Joseph Nazari, Glendale, CA (US)

(21) Appl. No.: 15/441,116

(22) Filed: Feb. 23, 2017

Related U.S. Application Data
(63) Continuation-in-part of application No. 15/067,132, filed on Mar. 10, 2016.
Application No. 15/067,132 is a continuation-in-part of application No. 13/825,759, filed on Mar. 22, 2013, now Pat. No. 9,314,312.
(60) Provisional application No. 61/685,545, filed on Mar. 20, 2012.
(60) Provisional application No. 61/685,752, filed on Mar. 22, 2012.
(60) Provisional application No. 61/690,106, filed on Jun. 19, 2012.

(51) Int. Cl. A61B 90/00; A61H 1/00

(52) U.S. Cl. CPC A61B 90/02; A61H 1/008

(57) ABSTRACT

A therapeutic device has an elevated or raised portion that is elastically deformable for releasable adherence to a patch of skin so as to lift the patch of skin above a sensitive underlying treatment site in reducing discomfort or pain associated with tissue compression at the treatment site. With the application of pressure to elastically deform the device from its original neutral configuration, the raised portion is brought into contact with the patch of skin, adhering the skin to the adhesive. When the pressure is removed, the device flexes with the raised portion exerting a lifting force on the attached patch of skin.

### CROSS-REFERENCE TO RELATED APPLICATIONS

This application is a continuation-in-part of U.S. patent application Ser. No. 15/067,132, filed on Mar. 10, 2016, which is a continuation-in-part of U.S. patent application Ser. No. 13/825,759, filed on Mar. 22, 2013, now U.S. Pat. No. 9,314,312, which is National Phase Patent Application and claims priority to and the benefit of International Application Number PCT/US2013/028099, filed on Feb. 27, 2013, which claims priority to and the benefit of U.S. Provisional Application No. 61/685,545, filed on Mar. 20, 2012; U.S. Provisional Application No. 61/685,752, filed on Mar. 22, 2012, and U.S. Provisional Application No. 61/690,106, filed Jun. 19, 2012, the entire contents of all of which are incorporated herein by reference.

### FIELD OF INVENTION

The present invention relates to a medical device for reducing symptoms of physical ailments and conditions, including pain and discomfort associated with Carpal Tunnel Syndrome (CTS), migraine headaches and other pressure-related ailments and conditions.

### BACKGROUND

Carpal Tunnel Syndrome (CTS) is idiopathic median neuropathy at the carpal tunnel of a human hand. With reference to FIG. 1, a human hand 100 is illustrated with muscles in a proximate portion, below thumb 106 and pinky finger 108, including abductor pollicis brevis 102 and abductor digit minimi 104, respectively. The muscles 102 and 104 define transverse carpal arch 110 with a recess 112 therebetween that is generally aligned with third finger 114. In tissues below the recess 102 is carpal tunnel 116 where median nerve 118 enters the hand 100. This tunnel is normally narrow as it is also occupied by flexor tendons 120, so any swelling of adjacent tissue, including the flexor retinaculum ligament 122, can pinch the nerve and cause pain, numbness, tingling or weakness.

The pathology of CTS is not completely understood but can be considered compression of the median nerve traveling through the carpal tunnel. Many studies have been conducted to identify causes but the evidence is not clear. Various results published in the 1990's found strong associations between the prevalence of CTS and forceful and repetitive wrist movements such as work on keyboard and computers. However, more recent research has cited genetics and obesity as possibly larger factors than use, and has encouraged caution in ascribing causality.

Pregnant women, especially those in their third trimester, may experience hand pain and discomfort, for example, numbness, tingling sensation, or aching in the hands and wrists, particularly at night when bodily fluids are more equally distributed throughout the body and not just in the lower extremities. Because pregnancy causes swelling in many parts of the body, extra pressure may be present on the nerves in the hands and wrists, including the median nerve.

The only scientific established disease modifying treatment is surgery to cut or divide the transverse carpal ligament. However, one study found that within two years of surgery, 75% of the patients showed recurrence of pain symptoms. Moreover, surgery has many risks and is not suitable for temporary CTS, such as that experienced by pregnant women. Palliative treatments for CTS include use of night splints and corticosteroid injection. Other nonsurgical treatment methods include hand braces, exercises, ergonomic equipment, oral diuretics and nonsteroidal anti-inflammatory drugs (NSAIDs). Websites including www.mycarpaltunnel.com discusses different treatment options. Other approaches include gloves, such as described in U.S. Pat. No. 6,006,751 and the prior art cited therein. Obviously, such gloves may be relatively expensive and are not disposable. Gloves may also limit manual dexterity and tactile sensitivity.

Kinesio tape has also been used as a nonsurgical treatment for CTS. Conventional kinesio tape is manufactured from a highly elastic strand wrapped in cotton fibers. It is employed as a gentle stretching therapy for soft tissue disorders and repetitive strain injuries. Kinesio tape has elasticity intended to match that of a person's skin, muscles, cartilage and connective fascia tissue. However, there are many different methods of taping around the hand and wrist and improper taping may cause further discomfort or even further damage to the median nerve. Moreover, proper taping often requires the use of two hands making it difficult if not impossible for a patient to apply the tape without assistance.

Hand braces and splints and temporary, removable devices worn on the hand are also known, for example, a stretching hand device described in U.S. Pat. No. 6,315,748 and sold under the mark THE CARPAL SOLUTION. The device includes a central, resilient, stretchable tensioning segment with a plurality of relatively less stretchable adhesive straps secured to the segment. In use, the segment is placed on the back of a patient's hand, whereupon the straps are pulled and adhered to the patient's palm in a fashion to expand the segment. In this orientation, the control segment exerts continuous yielding or tensile forces through the straps which in turn reduces carpal tunnel syndrome nerve compression and alleviates symptoms.

Another severe condition suffered by a significant portion of the population is migraine headaches. The pain of migraine occurs when excited brain cells trigger the trigeminal nerve to release chemicals that irritate and cause swelling of blood vessels on the surface of the brain. These swollen blood vessels send pain signals to the brainstem, an area of the brain that processes pain information. The pain of migraine is referred pain that is typically felt around the eye or temple area.

In addition to prescription and over-the-counter drugs, there are many remedies for migraines, including sleeping, taking caffeine, ice packs, and avoiding sunlight or certain foods. Kinesio tape has also been used to alleviate severe headaches. Experts believe that taping can significantly help reduce headaches by repositioning the muscles that are under stress and therefore helping to relieve tension in the neck.

Indeed, the adhesive tape or strips have many medical uses and applications beyond bandaging wounds. For example, nasal snoring strips are pieces of elastic plastic that are embedded in a sticking plaster for attachment across the outside of the nose. Shape-memory causes the plastic to straighten which effectively widens the nasal passages to allow for improved airflow. These strips are disposable and simplistic in design, construction and use.

Accordingly, there is a desire for a device that can reduce the symptoms of pressure-related conditions and ailments, including CTS and migraines. There is also a desire for the device to be simplistic in design, construction and use and be able to lift skin above a sensitive underlying site.

### SUMMARY OF THE INVENTION

The device of the present invention is primarily intended for use in those suffering from Carpal Tunnel Syndrome (CTS), but also has application for relieving migraine headaches and other situations where lifting the skin and tissue above a sensitive underlying site would provide benefits.

The present invention provides for an inexpensive, reliable and easy to use device which lifts an area of skin upwardly in a direction generally perpendicular to the skin surface. It does not interfere with the sleep of the user and is a low cost disposable item. In some embodiments, used for CTS, the device comprises a thin, generally rectangular member with a long dimension and a short dimension. The member is made from a plastic material, with “shape memory”, which in its normal configuration is generally flat. When pressure is applied orthogonally to the member, the member deforms. When released from pressure, the “shape memory” member has spring and rebounds to its normal configuration as much as possible. On a bottom (skin-facing) surface of the device, adhesive or adhesion member (used interchangeably herein) is provided on at least part of a central portion. A removable protective cover (e.g., release liner of wax paper) covers the adhesive.

In use, the protective cover is removed and the adhesive is applied to the palm proximate the wrist by a force depressing the center portion of the member toward the palm and fixes the center portion of the member to the palm. The member is oriented with the long dimension of the member across the palm between the thumb and the pinky finger. Since the adhesive is applied to a concave portion of the palm, the device forms a bridge across the palm. Side portions of the member supporting the center portion are not adhered to the palm so they can slide freely in contact with the palm. The skin of the palm adhering to the adhesive of the member is pulled upwardly as a result of negative pressure when the member is released and rebounds with its “shape memory”.

The adhesive can be applied to the device by any conventional means so that the adhesive remains on the member when the protective backing is removed. Alternatively, the adhesive can be applied by a user directly to the member or to the palm or skin of the user. Lifting and/or stretching the skin in the area of the palm or treatment site relieves symptoms, including the symptoms of CTS.

While some embodiments envision use of a single piece of spring-type plastic material, it is possible to incorporate other designs to provide a resilient or shape-memory member, such as synthetic whalebone, polyester boning material, metal, metal alloys, such as nitinol, and the like.

Accordingly, in some embodiments of the present invention, a therapeutic device adapted for application to skin covering a treatment site on a patient's body, has a generally rigid but elastically deformable body with at least one support portion and a bridge portion, the support portion adapted to rest on the skin, the bridge portion having an adhesive adapted to adhere to at least a portion of the skin, wherein the body is adapted to assume a neutral configuration with the adhesive and the portion of the skin separated by an air gap, and an adhesion configuration with the portion of the skin adhering to the adhesive being lifted and/or stretched at the treatment site. The body is also adapted to assume a depressed configuration the wherein the body is deformed from the neutral configuration with the bridge portion being depressed to bring the adhesive into contact with the portion of the skin.

The body while in its neutral configuration supports the bridge portion at a highest elevation relative to the skin surface of the treatment site, where as the body while in its depressed configuration supports the bridge portion at a lowest elevation. Moreover, the body while in its adhesion configuration supports the bridge portion at an in-between elevation greater than the lowest elevation and lesser than the greatest elevation. The adhesive provided on an inner, skin-facing surface may include a coating or layer of adhesive material, double sided adhesive tape, at least one suction cup, and/or micro-suction cup tape.

In more detailed embodiments, the therapeutic device has a thin sheet body with a “butterfly” configuration wherein the support portion supporting the bridge portion includes side planar portions adapted to rest on the skin. The butterfly configuration is well-suited for application to a palm of the patient's hand with the device positioned on the palm and the bridge portion longitudinally aligned with a median nerve of the palm. The adhesive is adapted for adhesion to skin below the device and above the median nerve.

The thin sheet body may also be configured rectangularly with rounded corners and one or more curved edges. As such, the device is also adapted for application on other areas of the patient's body. The rounded corners are atraumatic and the curved edge(s) accommodate features, curves and contours of the face or body, for example, nose, eyes, knuckles, elbows, knees and ankles.

In some detailed embodiments, the therapeutic device has a thin sheet preformed body having an outer surrounding rim and a raised center bridge portion. The rim and the bridge portion may have a common shape, for example, circular, rectangular, square or polygonal. The bridge portion is supported by at least three legs extending upwardly from the rim.. The preformed body is well-suited for application to a forehead, e.g., a temple region, of the patient. Each leg may be L-shaped including a radial leg portion and an axial leg portion. The radial leg portion extends between the bridge portion and the axial leg portion, and the axial leg portion extends between the radial leg portion and the outer rim.

In other embodiments, a therapeutic device has an elastically deformable sheet body having an elevated dome and a support portion, the elevated dome having an inner surface, and an adhesive on the inner surface, wherein the support portion has a base configured to contact a user's skin and the dome is configured to deform after adherence of the adhesive to a portion of the skin.

In some detailed embodiments, the dome has a first elevation and is configured to deform to a second elevation after adherence of the adhesive to the portion of the skin.

In some detailed embodiments, the dome after deformation is configured to pull on the skin after adherence of the adhesive to the portion of the skin.

In some detailed embodiments, at least a portion of the dome is configured to deform into a dimple after adherence of the adhesive to the skin.

In some detailed embodiments, the support portion includes at least one leg.

In some detailed embodiments, the support portion includes an annular flange.

In some detailed embodiments, a plurality of legs support the elevated dome.

In some detailed embodiments, the plurality of legs ranges between about two and five.

In other embodiments, a therapeutic device has an elastically deformable sheet body having a dome and a base, the dome having an inner surface, and an adhesive on the inner surface, wherein the dome is configured to deform into a dimple around the adhesive.

In some detailed embodiments, the base has a generally circular shape.

In some detailed embodiments, the adhesive has a generally circular shape.

In some detailed embodiments, the base has a diameter ranging between about 1.5 inches and 10 inches.

In some embodimenbts, a therapeutic device has an elastically deformable body with an elevated portion, a support portion, and an inner surface defining an inner volume of air. The device also has an adhesive on the inner surface. The body is adapted to assume a deformed configuration with a vacuum in the inner volume of air when subjected to an external force depressing the elevated portion.

In some detailed embodiments, the body has a generally shallow frusto-conical configuration.

In some detailed embodiments, the support portion has a skirt configuration.

In some embodiments, the elevated portion has a greater thickness and the support portion has a lesser thickness.

In some embodiments, the body of the device may have one or more predetermined curvatures in one or more directions. The curvature(s) serve to ensure that the bridge portion is elevated and separated from the skin below by an air gap when the device in its normal or neutral configuration. The curvature(s) also serve to strengthen the bridge portion against the pull of the adhered skin when the device has been depressed and applied to the treatment site and allowed to rebound to or toward its neutral configuration under its body's shape memory. In a more detailed embodiment, the bridge portion of the device has a curvature or concavity in at least one direction toward the inner, skin-facing surface.

The device may also be configured as a flexible band or sleeve or semi-rigid cuff to be worn on an appendage. The body is adapted to encircle or wrap around the appendage, e.g., an arm or a leg, with an inner, skin-facing surface having at least two projections or ridges with an adhesive positioned therebetween for adhesion to the skin, wherein the skin is lifted when adhered to the adhesive.

In other embodiments of the present invention, a method and system for manufacturing the aforementioned therapeutic device includes:

1. A sheet of plastic material is passed through a heating tunnel to flatten or smooth the material.

2. The material is passed through rollers, for example, silicone coated rollers, to curve the material with a desired radius.

3. The material is passed through an adhesive applicator which applies adhesive, including adhesive tape, with or without a release liner.

4. The material is passed through a cutting chamber, for example a die set, which cuts bodies of the devices out of the material.

Sequence of the above steps may be varied as desired or appropriate.

In some embodiments, the method of manufacturing includes custom-fitting the therapeutic device by making a mold of a body part to be fitted with the device and customizing the die set to the mold.

### BRIEF DESCRIPTION OF THE DRAWINGS

These and other features and advantages of the present invention will be better understood by reference to the following detailed description when considered in conjunction with the accompanying drawings. It is understood that selected structures and features have not been shown in certain drawings so as to provide better viewing of the remaining structures and features.

FIG. 1 is a perspective view of a hand with a cross-sectional view of a proximal portion including a median nerve and a carpal tunnel.

FIG. 2A is a top plan view of a therapeutic device, in accordance with one embodiment of the present invention.

FIG. 2B is a bottom plan view of the therapeutic device of FIG. 2A.

FIG. 2C is a lateral cross-sectional view of the therapeutic device of FIG. 2A, taken along line C-C.

FIG. 2D is a longitudinal cross-sectional view of the therapeutic device of FIG. 2A, taken along line D-D.

FIG. 3 is a top plan view of the therapeutic device of FIG. 2A, as applied to a user's palm.

FIG. 3A is a cross-sectional view of the therapeutic device of FIG. 2A, in a neutral configuration.

FIG. 3B is a cross-sectional view of the therapeutic device of FIG. 2A, in a depressed configuration.

FIG. 3C is a cross-sectional view of the therapeutic device of FIG. 2A, in an adhesion configuration.

FIG. 4A is a top plan view of a therapeutic device, in accordance with another embodiment of the present invention.

FIG. 4B is a bottom plan view of the therapeutic device of FIG. 4A.

FIG. 4C is a lateral cross-sectional view of the therapeutic device of FIG. 4A, taken along line C-C.

FIG. 4D is a longitudinal cross-sectional view of the therapeutic device of FIG. 4A, taken along line D-D.

FIG. 5A is a top plan view of a therapeutic device, in accordance with another embodiment of the present invention.

FIG. 5B is a bottom plan view of the therapeutic device of FIG. 5A.

FIG. 5C is a lateral cross-sectional view of the therapeutic device of FIG. 5A, taken along line C-C.

FIG. 5D is a longitudinal cross-sectional view of the therapeutic device of FIG. 5A, taken along line D-D.

FIG. 6 is a top plan view of the therapeutic devices of FIGS. 4A and 5A, as applied to a forehead of a user.

FIG. 7 is a schematic diagram of a system implementing a method of manufacture in accordance with an embodiment of present invention.

FIG. 7A is a perspective view of a machine system for manufacturing and packaging the therapeutic device of FIG. 2A, in one stage of operation in accordance with an embodiment of the present invention.

FIG. 7B is a perspective view of the machine system of FIG. 7A in another stage of operation in accordance with an embodiment of the present invention.

FIG. 8A is a perspective view of a therapeutic device, in accordance with another embodiment of the present invention.

FIG. 8B is a top plan view of the therapeutic device of FIG. 8A, in a neutral configuration.

FIG. 8C is a bottom plan view of the therapeutic device of FIG. 8A.

FIG. 8D is a cross-sectional view of the therapeutic device of FIG. 8B, taken along line D-D.

FIG. 8E is a cross-sectional view of the therapeutic device of FIG. 8A, in a depressed configuration, as applied to skin.

FIG. 8F is a cross-sectional view of the therapeutic device of FIG. 8A, in an adhesion configuration, as applied to skin.

FIG. 8G is a cross-sectional view of a therapeutic device, in accordance with another embodiment of the present invention.

FIG. 9 is a top plan view of the therapeutic device of FIG. 8A, as applied to a temple region of a user.

FIG. 10 is a schematic diagram of a system for implementing a method of manufacturing a therapeutic device in accordance with another embodiment of the present invention.

FIG. 11 is a perspective view of a therapeutic device in accordance with another embodiment of the present invention.

FIG. 12 is a perspective view of the devices of FIGS. 11 and 14, as applied to forearm of a user.

FIG. 13 is a perspective view of the device of FIG. 11, as applied to a thigh and a foot of a user.

FIG. 14 is a perspective view of a therapeutic device in accordance with another embodiment of the present invention.

FIG. 15 is a perspective view of a therapeutic device in accordance with another embodiment of the present invention.

FIG. 16 is a bottom perspective view of the therapeutic device of FIG. 15.

FIG. 17 is a perspective view of a therapeutic device in accordance with another embodiment of the present invention.

FIG. 18 is a perspective view of a therapeutic device in accordance with another embodiment of the present invention.

FIG. 19 is a perspective view of the therapeutic device of FIG. 15, with a dimple.

FIG. 20 is a detailed side cross-sectional view of a support portion with a beveled base.

FIG. 21 is a top plan view of the therapeutic device of FIG. 18, as applied to a user's palm.

FIG. 22A is a cross-sectional view of the therapeutic device of FIG. 21, in a neutral configuration, taken along line Z-Z.

FIG. 22B is a cross-sectional view of the therapeutic device of FIG. 21, subject to a force depressing the device into a deformed configuration.

FIG. 22C is a cross-sectional view of the therapeutic device of FIG. 21, deformed in an adhesion configuration.

FIG. 23 is a top plan view of therapeutic device of FIG. 15 applied to a user's temple.

FIG. 24 is a top plan view of the therapeutic device of FIG. 18 applied to user's forehead.

FIG. 25 is a side cross-sectional view of the therapeutic device of FIG. 18.

FIG. 26A is a perspective view of a therapeutic device in accordance with another embodiment of the present invention.

FIG. 26B is a top plan view of the therapeutic device of FIG. 26A.

FIG. 27 is a perspective view of a therapeutic device in accordance with another embodiment of the present invention.

FIG. 28 is a side cross-sectional view of the therapeutic device of FIG. 25, in a neutral neutral configuration.

FIG. 29 is a side cross-sectional view of the therapeutic device of FIG. 25, deformed in an adhesion configuration.

### DETAILED DESCRIPTION OF THE INVENTION

The present invention is directed to a device having a bridge portion adapted for releasable adherence to a patch of skin so as to lift the patch of skin above a sensitive underlying treatment site in reducing discomfort or pain associated with tissue compression at the treatment site. The device is positioned on the patient such that the bridge portion is elevated above the treatment site in a direction generally perpendicular to the patch of skin with an exposed adhesive facing the patch of skin to be lifted and/or stretched outwardly. With the application of pressure to elastically deform the device from its original neutral configuration, the bridge portion is brought into contact with the patch of skin, adhering the skin to the adhesive. When the pressure is removed, the device rebounds and generally reassumes its original configuration with the bridge portion lifting the attached patch of skin as it returns to an elevation at or near its original elevation.

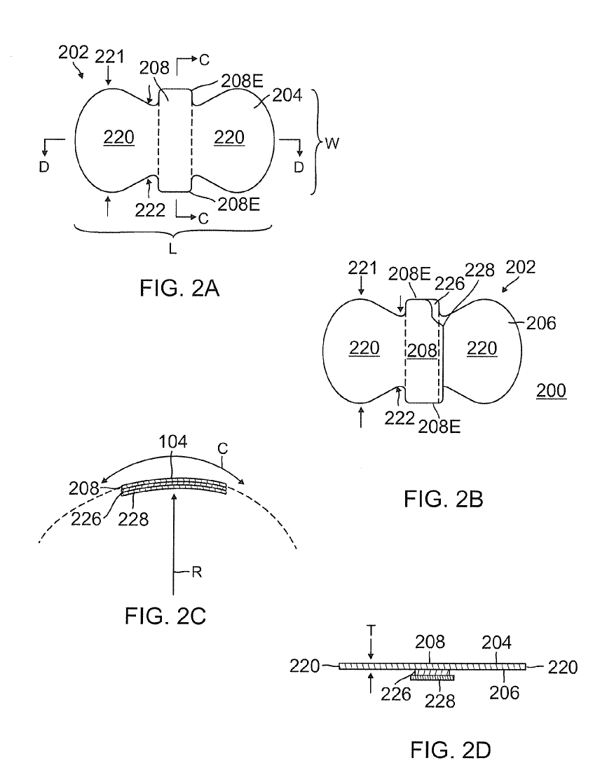

With reference to FIGS. 2A-2D, a device 200 has a thin sheet body 202 with a longer length dimension L and a shorter width dimension W. The body 202 has an outer surface 204 and an inner (skin-contacting or skin-facing) surface 206. The body 202 may be constructed of any suitable material with “memory” such that the body can be deformed under application of a force or load from an original shape or configuration and elastically return to the original shape or configuration after removal of the force or load. Depending on the underlying material of which the body 202 is constructed, the body 202 may have a thickness T ranging between about 0.012 and 0.018 inches (0.30 mm-0.45 mm), and preferably about 0.015 inches (0.40 mm). The body 202 may also be provided with one or more predetermined curvatures C, such as concavity or convexity, in one or both axes L and W.

Suitable materials for the body 102 include materials that are biocompatible or FDA-approved plastics, acrylate, polymers, metal, such as aluminum or stainless steel, or even wood or wood composites. A presently preferred material is polyethylene terephthalate (PET).

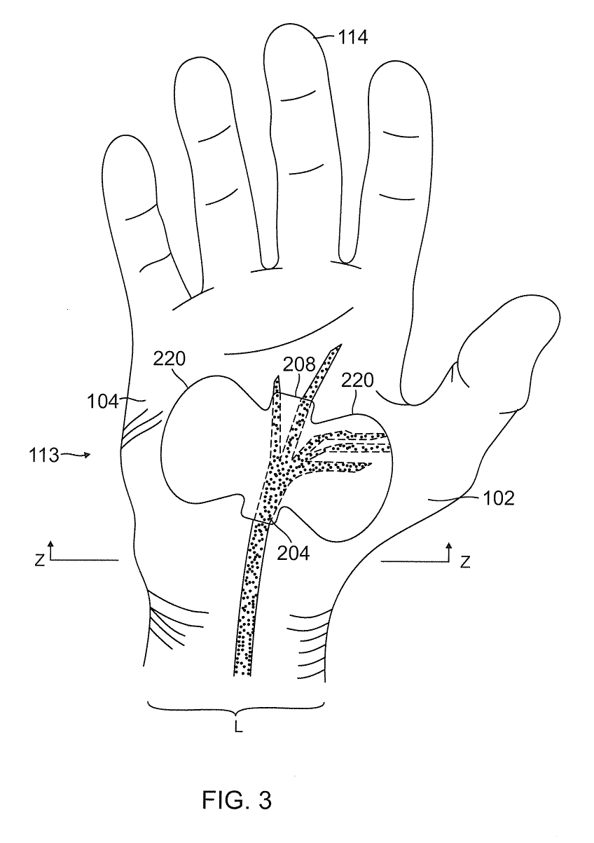

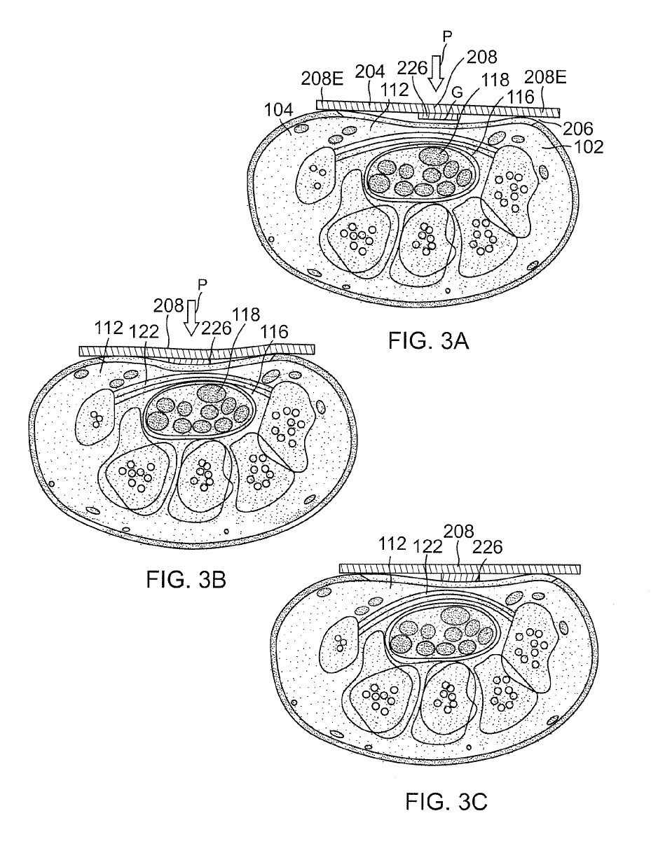

For use of the device 200 to treat the symptoms of CTS, the thin sheet body 202 of the device has a “butterfly” configuration adapted affixation to a palm 113 of a user, as illustrated in FIG. 3. The body 202 of the device 200 has two larger side support portions or “wings” 220 and an elevated bridge portion 208 extending between the support portions 220. The body 202 spans between about 1½ inches and 1¾ inches, and preferably about 1¼ inches in the direction of the shorter/width dimension or axis W, and about 2½ inches and 2¾ inches, and preferably about 2¼ inches in the direction of the longer/length dimension or axis L.

In other embodiments, the body 202 has a thickness T ranging between about 0.012 and 0.016 inches, and spans between about 1 inch and 1⅓ inches, and preferably about 1⅕ (1.235) inches in the direction of the shorter/width dimension or axis W, and about 1⅘ inches and 2.0 inches, and preferably 1 9/10 (1.900) inches in the direction of the longer/length dimension or axis L.

The bridge portion 208 is rectangular, being elongated along the axis W and spanning about 3/8 inch in the L axis. Each of the side portions 220 has a rounded “petal” shape with a greater outer width 221 that tapers slightly to a lesser inner width 222 adjacent the bridge portion 208.

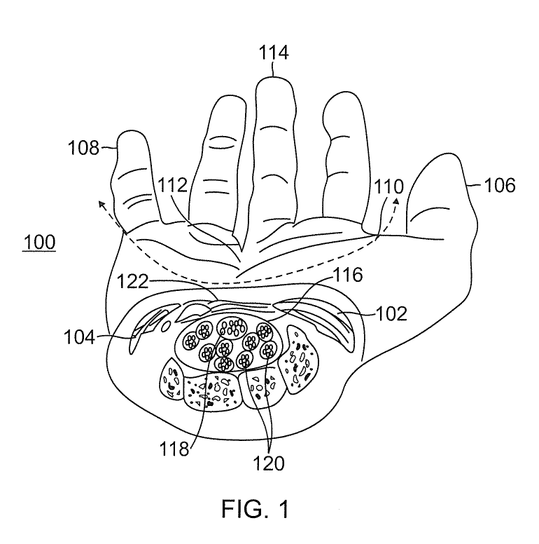

Moreover, the body 202 is provided with at least one predetermined curvature relative to an axis to conform to contours of the palm 113 near the wrist and location of the carpal tunnel (FIG. 1). In the illustrated embodiment of FIGS. 2C and 3, the body 202 has a predetermined concavity in the axis W toward the inner surface 206 to correspond with the convexity at the proximal end of the palm 113 (FIG. 1). The predetermined concavity of the body relative to the axis W may generally trace a circle defined by a radius R ranging between about 4 to 6 inch diameter, preferably about a 5 inch diameter.

The inner surface 206 of the bridge portion 208 is provided with an adhesion member or adhesive 226, for example, a coating or layer of adhesive for releasably affixing the device 200 to the palm. The adhesive 226 is covered and protected by a release liner 228 that is removed prior to use of the device 200 and application onto the user's skin.

With reference to FIGS. 3 and 3A-3C, use of the device 200 is described as follows. After the release liner 228 has been removed to expose the adhesive 226, the device 200 is placed over the palm 113 with the inner surface 106 and the adhesive 226 facing the palm 113. The device 200 is oriented with its longer axis L being generally transverse to middle finger 114. As shown in FIG. 3A, the device is positioned with each wing portion 220 resting on a respective convexity of the palm near muscles 102 and 104. With the device in its original and neutral configuration, the bridge portion 208 spans linearly in between the muscles 102 and 104, and elevated from the recess 112 and the median nerve 118. Advantageously, the slight but predetermined concavity of the device 100 in the lateral or W axis (FIG. 2C) enables the device to conform to the convexity of the palm 113, with the bridge portion 208 being elevated and separated from the skin below by an air gap G. As shown in FIG. 3B, to apply the device, pressure P is then applied, for example, by the user's other hand or that of an assistant, to the outer surface 204 of the bridge portion 208 above the adhesive 226 toward the skin so as to deform the bridge portion 208 (and any other portions of the body 202), bring the adhesive 226 into contact with the skin and allow the adhesive 226 to adhere to the skin, reducing or eliminating the air gap G.

When the pressure P is removed and the body 202 is released, the elastically deformable body 202 generally returns to its original neutral state in reassuming the predetermined concavity and the linearity between the bridge portion 208 and the wing portions 220, with at least a portion of the skin of the recess 112 adhering to the adhesive 226, as shown in FIG. 3C. Advantageously, the linearity or “bridging” of the bridge portion 208 when the body 202 rebounds pulls the adhered skin and adjacent tissue in the region of the recess 112 outwardly or away from the median nerve 118 to result in negative pressure and reduction of compression of the median nerve. In accordance with a feature of the invention, the lifted skin or tissue may be raised or repositioned from its original position a distance ranging between 1.0 mm to 5 mm, preferably 2.0 mm to 4.0 mm, and more preferably about 3.0 mm. To that end, the concavity of the body 202 which extends across the bridge portion 208 further strengthens the bridge portion 208 when lifting the skin against the pull of adjacent skin. Moreover, opposing ends 208E of the portion 208, each with three sides unattached to the body 102, allow the device 200 to be more flexible and comfortable while affixed to the palm, while providing additional bridging strength to the bridge portion 208 against the pull of adjacent skin, as well as providing additional surface area for the adhesive 226 to adhere with the skin.

In the disclosed embodiment, the bridge portion 208 is adhered to the skin, whereas the wing portions 220 are without adhesive and are not attached but are free to move and slide relative to the skin. This freedom of movement increases comfort to the user of the device by minimizing pinching or poking that may otherwise resulting discomfort if the wing portions (or any portions thereof) were also adhered to the skin. In that regard, a layer of fabric or liner or a coating of friction reducing gel may be provided on the inner surface of the wing portions 220 to prevent chafing. However, it is understood that the adhesive and adhesion member may be applied to one or more regions on the inner surface of the body and configured to cover beyond the bridge portion, for example, all or portion(s) of each or both wing portions 220.

It is understood that the bridge portion 208 need not be centered between the wing portions 220. That is, the size and/or shape of each wing portion 220 need not be equal or matching to the other. One wing may be larger, wider and/or longer than the other, as need or appropriate. There need not be symmetry about the axis L or W.

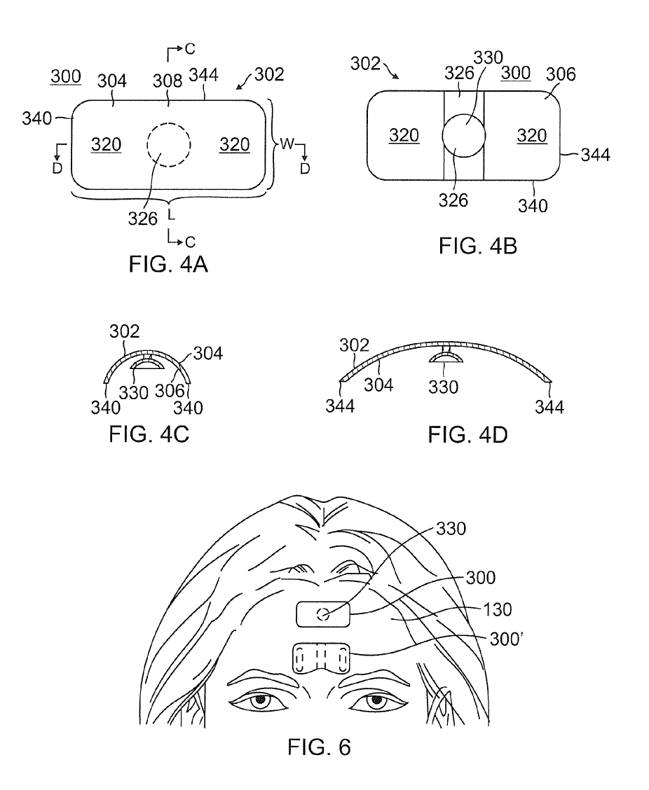

In an alternative embodiment of the present invention, a device 300 to treat the symptoms of a migraine headache is illustrated in FIGS. 4A-4D. Similarities between the devices 200 and 300 (as well as other devices described herein) may be denoted by similar reference numerals. However, each device has different adaptations for application and affixation to the patient's body. For example, the device 300 has adaptations for application and affixation to a generally flat skin site such as forehead 130 (FIG. 6), although it is understood that the device 200 may also be used to treat migraine headaches. A body 302 of the device 300 has a generally rectangular configuration with rounded corner 340, and a bridge portion 308 contiguous with two opposing rectangular or square side support portions 320. The body 302 spans between about ¾ inches and 1¼ inches, and preferably about 1.0 inches in the direction of the shorter/width dimension or axis W, and about 2.0 inches and 2½ inches, and preferably about 2¼ inches in the direction of the longer/length dimension or axis L. The bridge portion 308 is rectangular, being elongated relative to the axis W and spanning about ⅜ inch in the L axis.

The body 302 has at least two predetermined curvatures (same or different degree of curvatures), one in the axis W (FIG. 4C) and another in the axis L (FIG. 4D), to provide concavity toward the inner surface 304 in at least two directions.

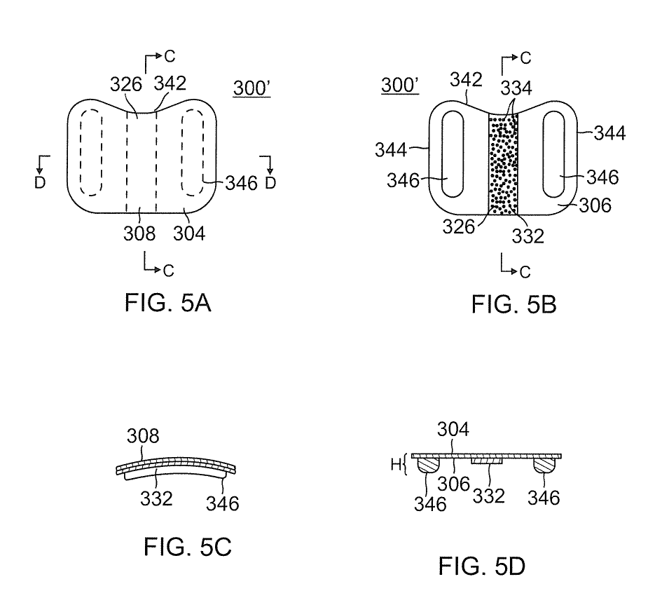

In another embodiment of the present invention for treatment of migraine symptoms, especially in the center of the forehead above the nose as illustrated in FIG. 6, a similar device 300′ of FIGS. 5A-5D may have one or more arcuate longitudinal edges 342 which can accommodate placement of the device around eyes, nose or ears.

For either device 300 or 300′, inner surface 306 of the bridge portion 308 is provided with adhesion member or adhesive, which may include one or more suction cups 330 (FIGS. 4A-4D) or micro suction cup tape 332 (FIGS. 5A-5D) having a surface with a multitude of microscopic craters 334 that work by creating many partial vacuums between the tape and target surface. The tape 332 can rebond repeatedly, and leaves no residue on the skin. A preferred tape is No. MSX-6800, DC Conformable Hi-Tack Type tape manufactured by 3M.

Moreover, for either device 300 or 300′, on the inner surface 306 at or near lateral edges 344, each side support portion 320 includes a projection, such as a raised ridge 346 with a height H (FIGS. 5A-5B) which elevates the body 302 away from the skin so that the bridge portion 308 forms a bridge that is separated from the skin with an air gap in between. In that regard, the body 302 has sufficient rigidity to provide linearity between the portions 308 and 320. Each ridge 346 may have a different height, depending on the contour of the site. The ridges 346 may be constructed of the same material as the body 302 or of any other suitable material that provides sufficient form and structure to lift the body 302. Where the body 302 spans about 1 7/16 inches along the axis L, and about 1 3/16 inches along the axis W, each ridge 346 may have a thickness of about 3/16 inch and a width ranging between about ⅛ inch and ¼ inch. Ends of each ridge may terminate a distance of about ⅛ inch from the longitudinal edges 340.

It is noted that the concavity/ies of the bodies (actual or as effectuated by the ridges 346) should be of a sufficient degree such that peripheral edges, including longitudinal edges 340 and lateral edges 344 extend beyond the adhesive (e.g., adhesive coating, adhesive tape, suction cups, or micro-suction cup tape) so as to elevate the adhesive from skin and provide an initial air gap before the skin is adhered to the device (as shown in FIGS. 4C, 4D and 5D).

In use, the adhesive 326 is exposed and the device 300′ is placed over the target skin site with the inner surface 306 and the adhesive 326 facing the skin. The device 300 is oriented with the ridges 346 straddling the skin site so that the bridge portion 308 and the adhesive 326 is directly over the sensitive skin site. Elevated by the ridges 346, the bridge portion 308 is situated above the skin, separated by an air gap. With the device so positioned, pressure is then applied by the user (or an assistant) to the outer surface 304 of the bridge portion 208 directly above the adhesive 326 toward the skin so as to deform the bridge portion 308 (and any other portions of the body 302), bring it into contact with the skin and allow the adhesive 326 to adhere to the skin.

When the pressure is removed and the body 302 is released, the elastically deformable body 302 generally returns to its original neutral state in reassuming the linearity between the bridge portion 308 and the side portions 320, thereby lifting the adhered skin and tissue and reducing compression at the site. The device can be situated around the eyes, near a temple, or above the nose on the forehead, as facilitated by the arcuate longitudinal edges 240, as shown in FIG. 6.

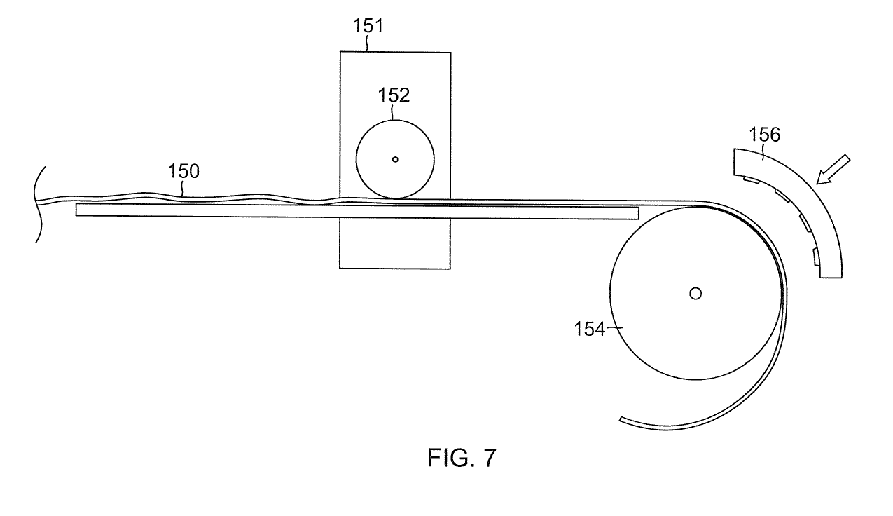

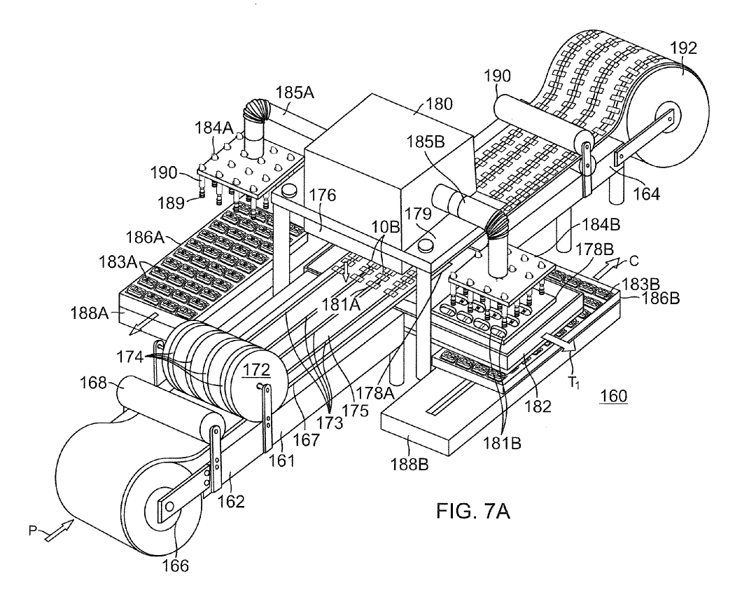

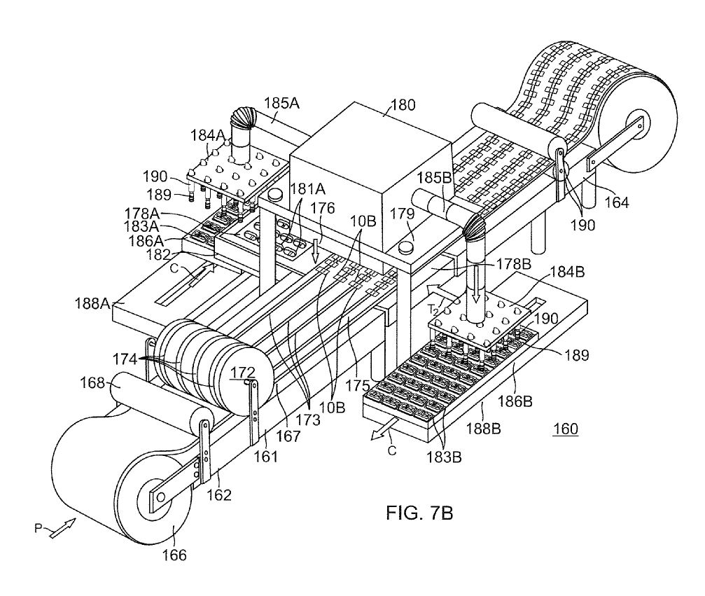

With reference to FIG. 7 , the foregoing devices may be manufactured in accordance with the following method:

1. Providing a single flat sheet 150 of suitable material;

2. Heating the sheet to smooth the sheet 150 and remove wrinkles and bends;

3. Stamping, die-cutting or otherwise cutting the sheet 150 to form the device.

4. Applying the adhesive to the inner surface of the bridge portion.

The method may include forming the sheet 150 with at least one predetermined curvature.

Heating the sheet may include passing the sheet 150 through a heat tunnel 151, and/or under or between heated roller(s) 152. Forming the sheet 150 with at least one predetermined curvature may include passing the sheet through heated roller(s) 154. Stamping, die-cutting or otherwise cutting the sheet into device bodies may be performed by a die set including roller 154 and a movable punch 156. The movable punch 156 may be curved to conform to the curvature of the roller 154 to simultaneously form the device body with at least one or more predetermined curvature. Applying the adhesive 326 may occur at any stage, including before, during or after stamping, die-cutting or otherwise cutting the sheet. As mentioned, the adhesive may be medical grade adhesive suitable for topical use. It may take the form of a solid coating or layer (or a plurality of adhesive lines), double-sided adhesive tape, suction cup(s) or micro-suction cup tape. Any release liner may be applied simultaneously with the application of the adhesive or afterwards.

Where the device has ridges 346, the die set may have formations to stamp the ridges into the sheet, or they may be mounted after stamping. Alternatively, the method of manufacture may include applying or adhering the ridges 346 to the inner surface of the bridge portion before, during or after application of the adhesive to the inner surface.

An embodiment of a machine system 160 implementing a method of manufacture and packaging of the device is illustrated in FIGS. 7A and 7B. The machine system 160 includes a longitudinal conveying platform 161 that defines a conveying pathway P from a first end 162 downstream to a second end 164. At or near the first end 162 is dispenser roller 166 of sheet material 167. Stored in roll form, the sheet material advantageously has a preformed curvature. To that end, the dispenser roller 166 in one embodiment of the invention has an initial diameter no greater than about 25 inches and more preferably no greater than about 20 inches.

Downstream of the dispenser roller 166 is a smooth roller 168 positioned to flatten the sheet material and remove wrinkles or bumps. Downstream of the smooth roller 168 is an adhesive tape applicator roller 172 which applies double-sided adhesive tape strips 174 onto an upward surface 175 of the sheet material (which becomes the inner/skin-facing surface of the device). The tape strips 174 are mounted on the applicator roller 172 with an exposed adhesive surface 171 facing outwardly so that it contacts and affixes to the upward surface 175 of the sheet material 167 upon pressure applied by the roller 172. Release liner strips 173 are affixed to the opposing surface of the applied tape strips 174 for subsequent removal by users when applying the devices.

Downstream of the applicator roller 172 is a die press 179 with upper platen 176 and lower platens 178A and 178B driven by air hydraulics assembly 180. The upper platen 176 is driven toward first lower platen 178A in a first stage operation of the die press and toward the second lower platen 178B in a second stage operation of the die press, as described below, to die cut a plurality of individual devices 10A and 10B from the sheet material 167 and the tape strips 174 affixed thereon.

The lower platens 178A and 178B are supported on a carrier 182 that is adapted for translational movement in opposition directions T1 and T2 along a pathway generally transverse to the conveying pathway P. In a first stage operation (FIG. 7A), the carrier 182 slides in direction T1 to position the first lower platen 178A directly below the upper platen 176 wherein the upper platen 176 engages the first lower platen 178A and cuts devices 10A in die recesses 181A, exposing the second lower platen 178B off one side of the conveying platform 161 and rendering it accessible to a transport member 184B. In the second stage (FIG. 7B), the carrier 182 slides in direction T2 to position the second lower platen 178B directly below the upper platen 176, wherein the upper platen 176 engages the second lower platen 178B and cuts devices 10B in die recesses 181B, exposing the first lower platen 178A off the opposite side of the conveying platform 161 and rendering it accessible to an opposing transport member 184A. Accordingly, the carrier 182 translates each lower platen 178A and 178B between an engagement position under the upper platen 176 and an unload position under respective a transport member 184A. Where a full die cut cycle of the die press involves the first stage operation followed by the second stage operation in sequence, the hydraulics assembly drives the die press 179 and carrier 182 in a manner whereby one of the lower platen is placed under the upper platen 176 for die cutting and while the other lower platen is exposed and accessible to a respective transport member 184. That is, in one stage of operation, the one lower platen is in the engagement position whereas the other lower platen is in the unload position, and in the other stage of operation, the other lower platen is in the engagement position whereas the one lower platen is in the unload position.

In addition to driving the die press 179 and the carrier 182, the hydraulics assembly 180 also drives support arms 185 to move the transport members 184A and 184B between at least three positions: a wait position, a load position, and an off-load position. The transport members are adapted to secure a load of devices and transport the devices from the lower platens 178A and 178B to packaging or dispensers 183 arranged on tray members 186A and 186B arranged below the lower platens in their exposed positions. As shown in FIG. 7A, when lower platen 178B is exposed and accessible to transport member 184B, the transport member 184 is actuated by the hydraulics assembly 180 to move from its wait position to the load position where it picks up devices 10B from the lower platen 178B. In the illustrated embodiment, the transport member 184 includes grippers with vacuum/suction cups 189 mounted on posts 190 which are activated to grab the devices 10B individually. With a load of devices on board, the transport member 184B is actuated by the hydraulics assembly 180 to move into the off-load position toward the tray member 186B positioned below the exposed position of the lower platen 176B and release the devices 10B into dispensers 183B. The transport member184B is then returned to its wait position for the next cycle when the lower platen 176B contains more cut devices and is exposed and accessible again.

The tray member 186B is supported on carrier 188B which translates the tray member 186B along direction C parallel with the conveying pathway P so that the tray member 186B can shift between different positions for efficient loading of the dispensers 184. In the illustrated embodiment, the tray member is translated between two positions: a proximal position and a distal position.

It is understood that the actuation and movement of the die press 179, the carrier 182, the transport members 184A and 184B and the carriers 188 may be coordinated and synchronized with each other, as desired or appropriate for efficient operation of the machine system. For example, the upper platen 176 may be driven to engage lower platen 178A as the support arm 185B is driven to pick up devices 10B from the lower platen 178B, and as the support arm 185A is releasing devices into the dispensers 181A. A suitable system and machine for transporting work pieces is described in U.S. Pat. No. 8,230,989, entitled SHUTTLE MACHINE FOR MACHINE TOOL, the entire contents of which are hereby incorporated by reference.

The spent sheet material 167 downstream of the die press 179 is drawn downstream by a servo drive rollers 190 and collected on a waste roller 192 at the second end 164 of the conveying platform 161.

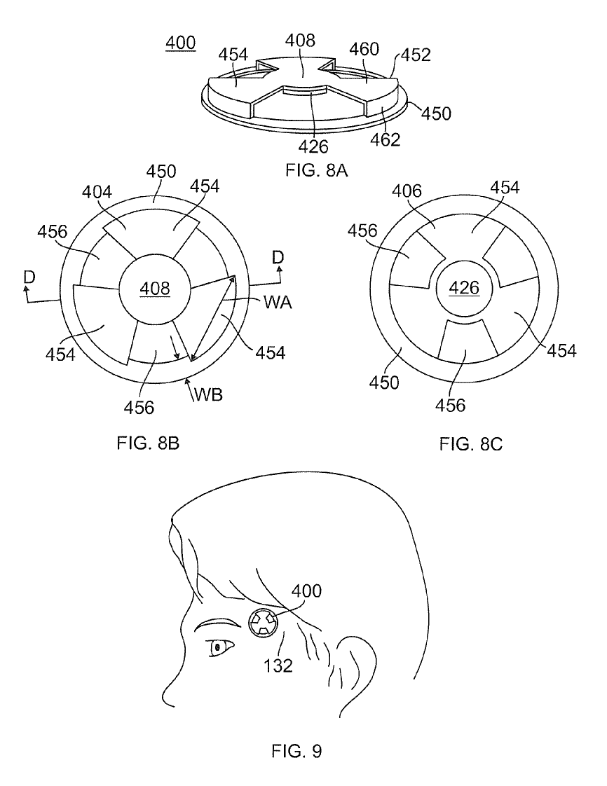

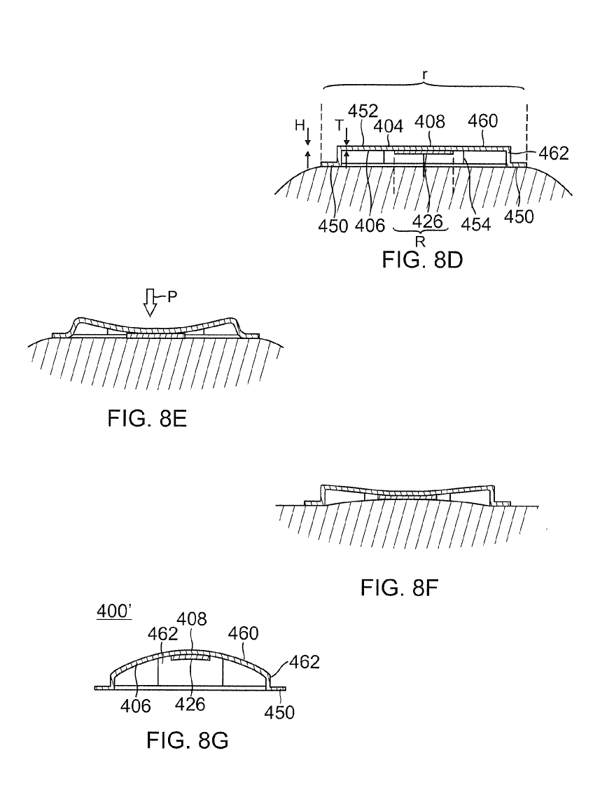

Another embodiment of the present invention is illustrated in FIGS. 8A-8C. A device 400 has a thin sheet body 402 which has been formed with a raised center profile. The body 402 has a support portion 420 and an elevated planar center bridge portion 408. The support portion 420 is adapted to rest on skin above a sensitive underlying site, such as temple 132, as shown in FIG. 9. The support portion 420 includes an outer rim or base 450 and radially extending L-shaped legs or posts 454 projecting therefrom that connect the base 450 and the bridge portion 408. The base 450 is adapted to rest on and generally surround skin of a sensitive underlying site, such as temple 132, as shown in FIG. 9. Between adjacent pairs of legs is an opening 456. As better seen in FIGS. 8D-8F, each leg has a radial portion 460 coplanar with the bridge portion 408 and an axial portion 462 extending between the radial portion and the base 450. The axial portion 462 may extend at an angle ranging between about 45 to 120 degrees, preferably about 60 to 100 degrees, and more preferably about 90 degrees, from the base 450. The plurality of legs 454 ranges between about two and five.

With reference to FIGS. 8A-8F, the illustrated embodiment of the device has three legs 454 positioned equidistance from each other (e.g., centered at about 0, 120 and 240 degrees), and three openings 456. Moreover, each leg presents a 90 degree angle between the radial portion 460 and the axial portion 462, and also between the axial portion 462 and the base 450. Width of the legs can vary depending on the plurality of legs and the desired flexibility of the bridge portion 408. An inner surface 406 of the bridge portion 408 has an adhesion member or adhesive 426, for example, a coating or layer of adhesive, an adhesive tape or a micro suction cup tape, as described above. An inner surface of the rim 450 may also be provided with an adhesion member or adhesive to help secure the device on the skin.

In one embodiment, the outer radius r of the base 450 is 1.375 inches, the radius R of the bridge portion 408 is 0.5 inch, the width WA of the axial portion 462 is 0.625 inch, and the width WB of the base 450 is 0.125 inch. The height H of the bridge portion 408 from the base 450 is 0.1875 inch and the thickness of the body 402 throughout the device 10 is 0.015 inch.

The base 450 and the bridge portion 408 may each be any shape, for example, rectangular, polygonal, oval, or circular. In the illustrated embodiment, both the base and the bridge portion are circular.

In use, the adhesive 426 is exposed and the device 400 is placed over the target skin site with the base generally surrounding the site and the adhesive 426 facing the skin and tissue, as shown in FIG. 8D. Elevated by the legs 454, the bridge portion 408 is situated above the skin, separated by an air gap generally equal to the height H. With the device so positioned, pressure P is applied by the user (or an assistant) to the outer surface 404 of the bridge portion 408 toward the skin so as to deform the upper body 452 (including the bridge portion 408 and/or any of the legs 454), bring the bridge portion 408 into contact with the skin and allow the adhesive 426 to adhere to the skin, as shown in FIG. 8E.

When the pressure is removed and the upper body 452 is released, the elastically deformable body 402 rebounds and generally returns to an elevation at or near its original elevation wherein the bridge portion 408 lifts the adhered skin and tissue and reducing compression at the site.

As shown in FIG. 8D, the bridge portion 408 and the radial leg portion 460 may extend perpendicularly relative to the axial leg portion 462, or the portions 408 and 460 may have at least one predetermined curvature in a direction. As illustrated in FIG. 8G, device 400′ has an upper body 402′ with curvature in more than one axial direction to provide a concavity toward the inner surface 406 that forms an elevated dome.

The devices of the present invention may be worn at all times, or only a specified time periods, such as night time, during sleep. Each device may be reused so long as the adhesive is functional. If not, the device may be discarded, or new adhesive may be applied to the device over or in place of the spent adhesive. Due to the inexpensive cost of the device and its components, the device may be economically discarded as trash or for recycling.

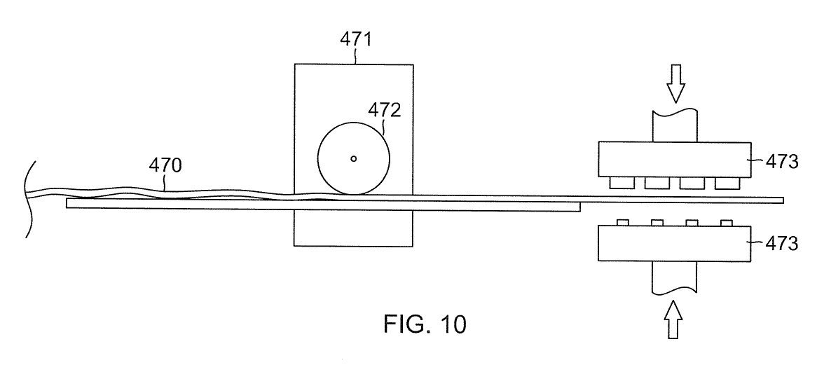

With reference to FIG. 10, the device 400 can be manufactured as follows:

1. Forming the device;
(a) Injection, press or vacuum forming the base 450, bridge portion 408 and the legs 454; or
(b) Providing a single flat sheet 470 of suitable material;
   (i) Heating the sheet to smooth the sheet 370 and remove wrinkles or bends;
      1. Heating by passing through a heat tunnel 471; and/or
      2. Heating by heated roller(s) 472.
   (ii) Stamping, die-cutting or otherwise cutting sheet to form the base 450, the bridge portion 408 and the legs 454;
      1. Die punching out the openings 456 to form the legs with die set 473;

2. Applying the adhesive 426 to the inner surface 406 of the bridge portion 408.

The stamping, die-cutting or otherwise cutting of the sheet may include both cutting of the base 450, bridge portion 408 and legs 454 from the sheet and reforming/reshaping to provide the curvature of the upper body 452 and/or angulation or bend in the leg 454 for elevating the bridge portion 408 from the base 450. The application of the adhesive 426 may occur before, during or after stamping, die-cutting or otherwise cutting of the sheet.

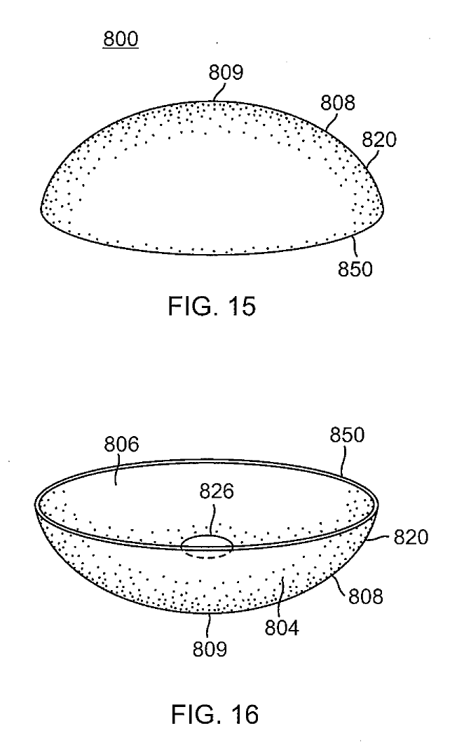

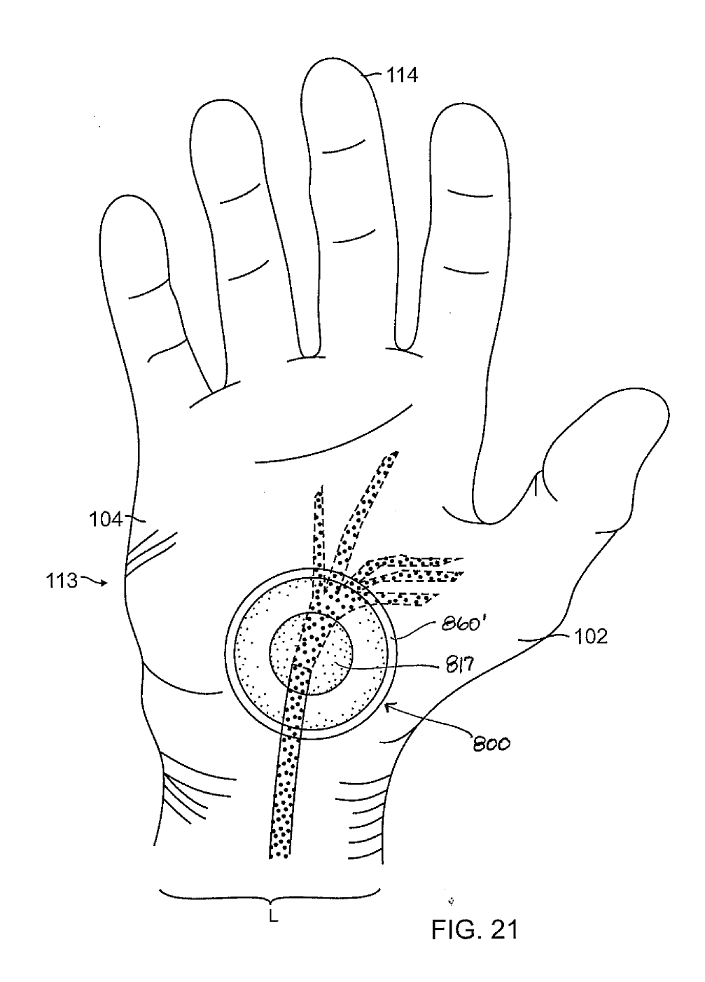

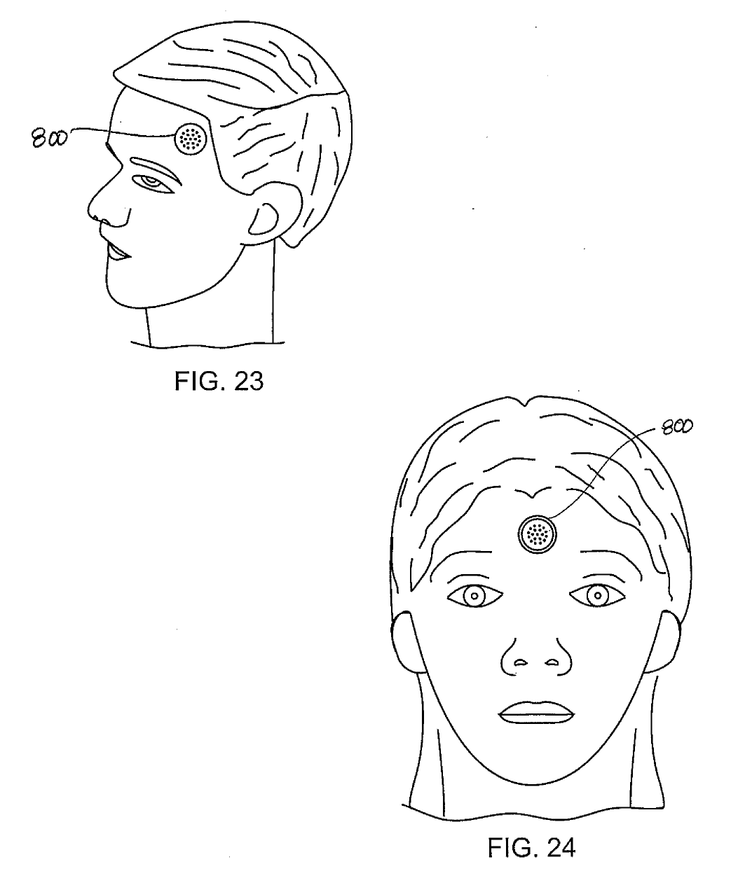

In some embodiments, a device 800 of the present invention has a thin sheet body 802 which has been formed with a support portion 820 and an elevated main portion or dome 808, as shown in FIGS. 15 and 16. The dome is defined by a concavity on an inner (skin-facing) surface 806 and a corresponding convexity on an outer surface 804. The support portion 820 is on the periphery of the main portion 808 has a base 850 with a generally circular shape, including, for example, a circle, oval or ellipse, and is adapted to make contact with and rest upon skin of a sensitive underlying site such as across the palm (FIG. 21), or on the temple (FIG. 23) or forehead (FIG. 24). In some embodiments, the base 850 is beveled, as shown in FIG. 20, so the base is generally free of sharp edges and presents a more atraumatic configuration against the user's skin for comfort. Having a generally dome configuration, the raised main portion 808 has a center apex portion 809 located at a maximum height relative to the peripheral support portion 820.

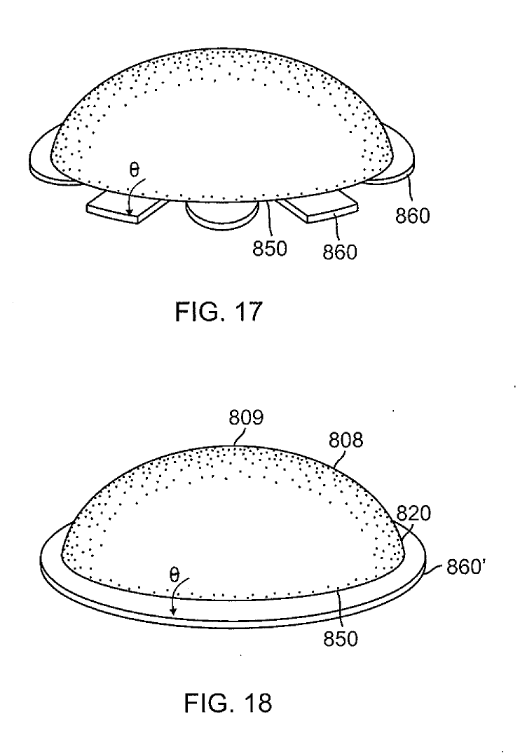

In some embodiments, as shown in FIG. 17, the body 802 includes one or more generally radial legs 860 that extend an angle θ from the base 850 of the peripheral support portion 820. The angle θ ranges between about 90 and 135 degrees, preferably about 110 degrees, so that the one or more flanges can lie flat against the skin and provide additional support to the body 802. In some embodiments, as shown in FIG. 18, the radial legs 860 are continuous forming an annular flange 860′ that extends around the peripheral support portion 802.

The body 802 is constructed of any suitable material, including, for example, biocompatible or FDA-approved plastics, acrylate, polymers, metal, such as aluminum or stainless steel, or even wood or wood composites. One suitable material is polyethylene terephthalate (PET), polypropylene or polyurethane. In some embodiments, the construction material is generally transparent.

As shown in FIG. 16, inner surface 806 of the device 800, in the region of or near the apex portion 809, includes adhesive 826, for example, an adhesive coating or layer, or an adhesive member, e.g., double-sided adhesive tape, suitable for releasable adherence to skin above a sensitive underlying treatment site that is to be lifted by the device 800 in reducing discomfort or pain associated with tissue compression at the treatment site. The adhesive 826 may have any suitable shape, including a circular shape, an elongated shape, a rectangular shape, a polygonal shape or even a donut shape. In some embodiments, the adhesive 826 is covered and protected by a release liner 828, for example, a thin sheet of paper or plastic, that is removed by a user prior to application of the device 800 onto the skin.

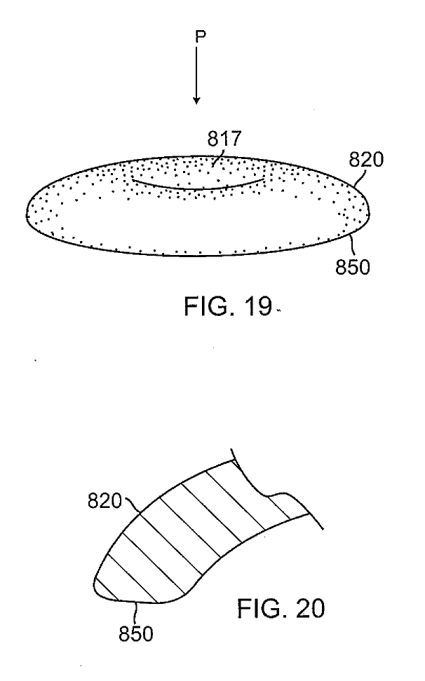

The thin sheet construction provides the body 802 with sufficient rigidity to maintain the dome configuration when the device 800 is in a neutral configuration, such as when there are no external force or pressure P applied to the device 800, as shown in FIGS. 15 and 18, yet allows the body 802 to elastically flex into a deformed configuration when an external normal force is applied onto the main portion 808, especially the apex portion 809, as shown in FIG. 19. In the deformed configuration, the support portion 803 generally maintains its shape and contact with the skin while the apex portion 809 inverts to form a dimple 817 in the main portion 808.

Where the external normal force is sufficient to result in adhesion of the adhesive 826 to the underlying skin after removal of the external normal force, the thin sheet construction provides the body 802 with resiliency so that it attempts to rebound into the neutral configuration with the adhesive 826 and the dimple 817 advantageously exerting a lifting force on the adhered skin. The size and shape of the dimple 817, and the lifting force exerted depends on a number of a factors, including, for example, the size and shape of the adhesive 826, the elasticity and flexibility of body 802, the elasticity of the skin, the concavity of the body 802, and the region of the user's body onto which the device is applied. The adhesive 826 may be circular, oval, elongated, rectangular, or any other suitable shapes regardless of the size and shape of the body 802.

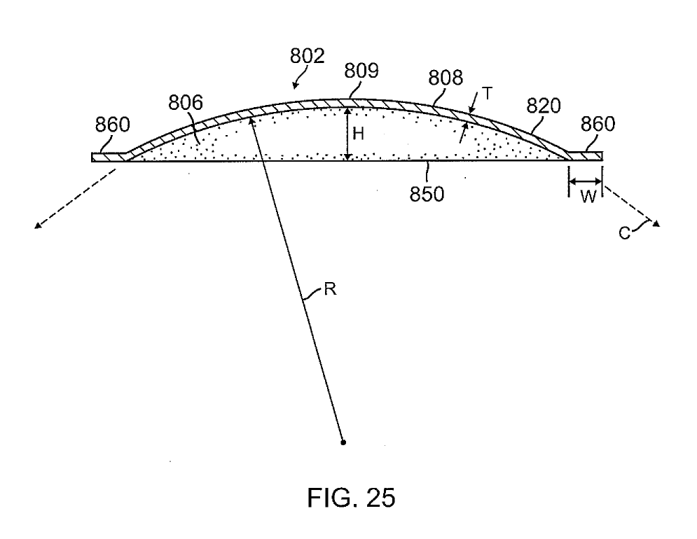

As shown in FIG. 25, in some embodiments, the body 802 has a generally uniform thickness T ranging between about 0.012 and 0.018 inches (0.30 mm-0.45 mm), and is preferably of about 0.015 inches (0.40 mm). In some embodiments of the device in its neutral configuration, the base 850 of peripheral support portion 820 spans a diameter D ranging between about 1½ inches and 2¾ inches. In some embodiments of the device, the one or more legs 860 or flange 860′ has a width ranging between about ½ inch and ⅜ inch. In some embodiments of the device in its neutral configuration, the apex portion 809 has a vertical height H ranging between about ½ inch and ⅜ inch. In some embodiments, the dome profile of the body 802 traces the arc of a circle C having a radius ranging between about 0.5 inch and 5.0 inches.

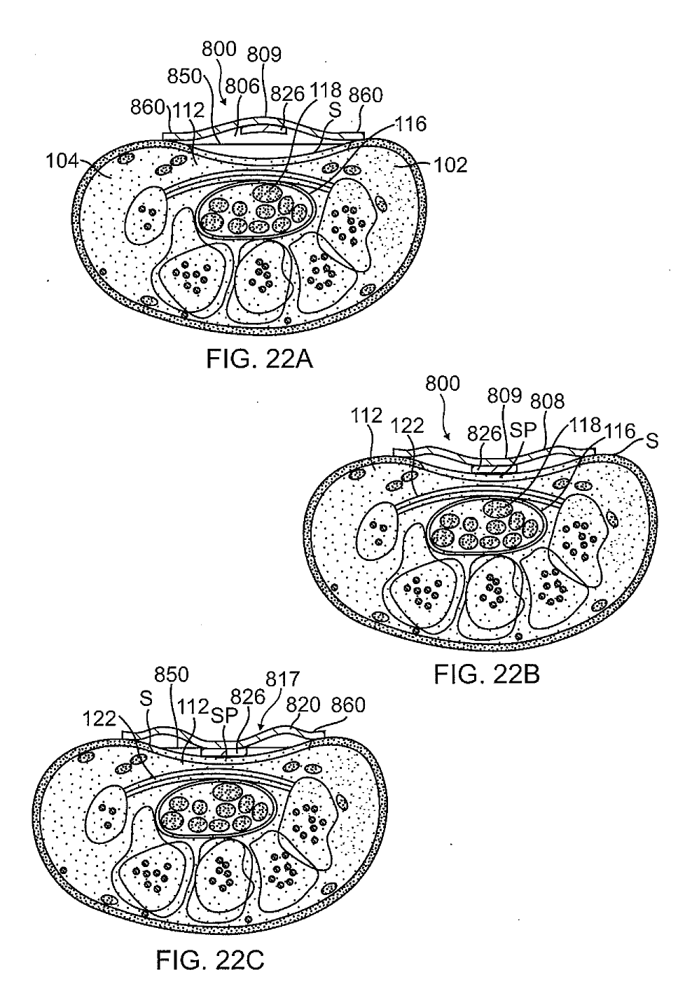

In use, the release liner 828 is removed to expose the adhesive 826. As shown in FIG. 22A, the device 800 in its neutral configuration is positioned on the user such that the inner surface 806 and the adhesive 826 face the skin S and the base 850 and any legs 860 or flange 860′ rest on the skin S. The apex portion 809 is positioned above the skin portion SP to be lifted or stretched outwardly. As shown in FIG. 22B, with the application of an appropriate external normal force or pressure onto the outer surface 804 in the region of the apex portion 809 depressing the apex portion 809, the body 802 elastically deforms with the apex portion 809 inverting and forming the dimple 817 and the adhesive 826 coming into contact with and adhering to the skin portion SP. When the user releases the apex portion 809 from the external normal force, the apex portion 809 attempts to rebound resulting in the adhesive 826 exerting a pull on the skin portion SP, as shown in FIG. 22C.

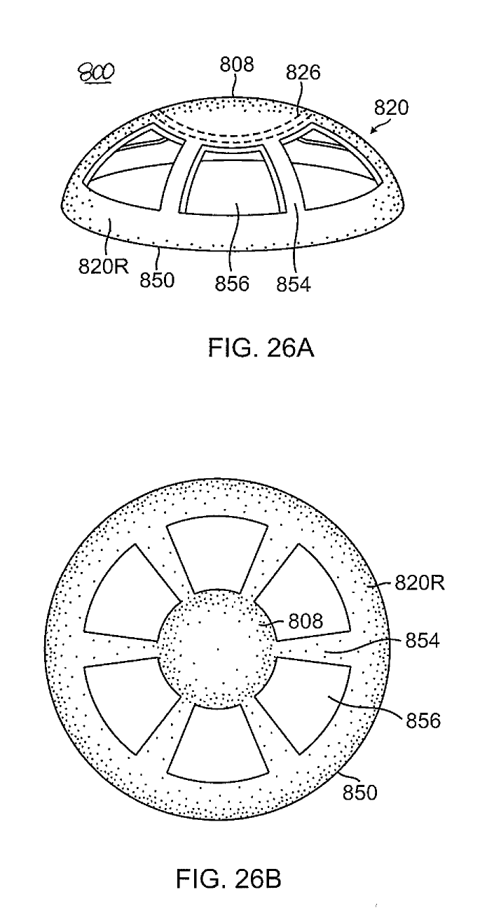

In another embodiment, the support portion 820 includes a plurality of deformable legs 854 separated by openings 856, as shown in FIG. 26A and FIG. 26B. The elevated portion or dome 808 is supported by the plurality of axial legs 854 that connect the elevated portion 808 and a radial band 820R of the support portion 820. The plurality of legs 854 ranges between about two and five, and they are generally equidistance from each other. Width of the legs can vary depending on the plurality of legs and the desired flexibility of the elevated portion 808. The adhesive 826 is provided on the inner surface of the elevated portion 808.

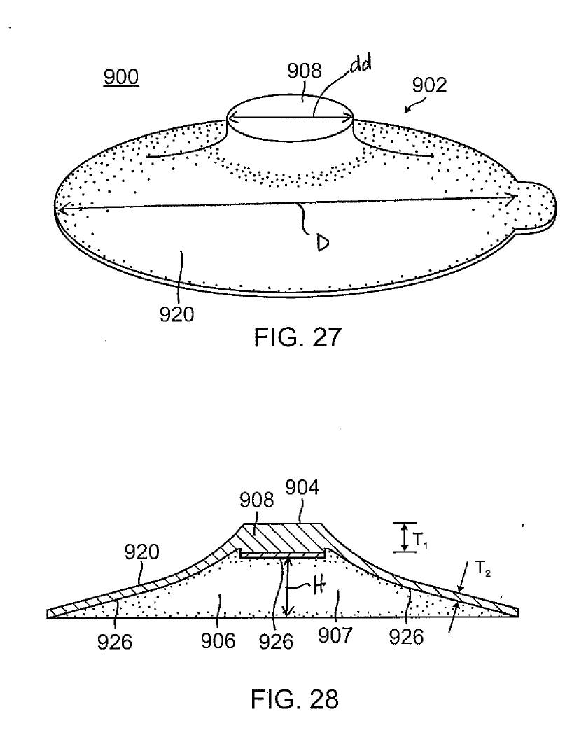

In FIG. 27, a device 900 of another embodiment includes an elastically flexible body 902 an having shallow frusto-conical shape with an elevated or raised portion 908 of a greater, generally uniform thickness T1 and a support portion 920 of a lesser, generally uniform thickness T2 configured as a flared skirt surrounding the elevated portion 908. The raised portion 908 has a generally circular cross-section and a generally flat upper surface 904. As shown in FIG. 28, an inner surface 906 is configured to face skin S on which the device 900 is adapted to rest with the skirt support portion 920 supporting the elevated, generally centered, portion 908 at an elevation above the skin when the device is in a neutral configuration. The skirt support portion 920 is configured to create an inner volume or cavity 907 of air defined between the inner surface 906 and the skin S. The inner surface 906 includes an adhesive 926 in the region of the elevated center portion 908 which is positioned over skin portion SP to be lifted by the device 900.

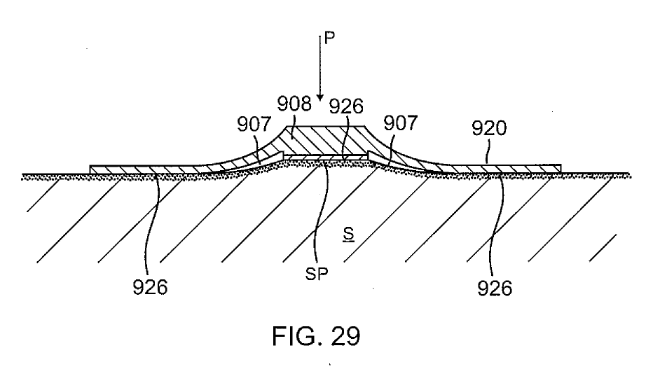

When subject to an external normal force or pressure P that is applied onto the upper surface 904 of the elevated portion 908 in the area of the adhesive 926, as shown in FIG. 29, the body 902 deforms with the skirt support portion 920 flattening and expelling the air from the inner cavity 907 in creating a vacuum in the reduced inner cavity 907. The greater thickness T1 of the elevated portion 908 enables the air in the inner cavity 907 to be effectively expelled out radially from under the skirt support portion 920 with the thinner thickess T2. At least a majority portion of the inner surface 926 comes into contact with the skin S and the adhesive 926 adheres to the skin portion SP to be lifted. With removal of the pressure P, the elastically flexible body 902 advantageously attempts to rebound back into the neutral configuration, thus exerting a lifting force on the skin portion SP. The adhesive 926 secures the device 900 and the skin portion SP to each other. The vacuum created in the inner cavity 907, particularly in the center of the device around the adhesive 926, as concentrated by the greater thickness T1 of the elevated portion 908, increases the lifting force on the skin portion SP. In some embodiments, the skirt support portion 920 includes a tab 913 by which a user can readily lift for removing the device 900 from the skin.

The device 900 is made of any suitable elastically flexible material, including, for example, soft, pliable injected plastic, such as polypropylene or polyurethane. The material may be translucent or transparent. In some embodiments, a diameter D of the device 900 ranges generally between about 0.75 inch and 2.0 inches, and a height H of the inner cavity 907 ranges generally between about 0.25 and 0.75 inch. With a shallow frusto-conical configuration, the ratio of height H to diameter D of the body 902 ranges generally between about 0.3 to 0.375. The ratio of a diameter or width dd of the raised center portion 908 to the diameter D of the device 900 ranges between about 0.2 and 0.33.

In yet another embodiment of the present invention, a device 500 may be worn as a band or sleeve encircling a limb or a portion thereof. With reference to FIG. 10, the device 500 is elongated defining a length axis L and a width axis W. The device 500 has a generally rigid yet flexible main body 511 having a planar, generally rectangular form, and two fastening side portions 513, each having a generally rectangular form and adjoined to an opposing side edge 515 of the main body 511. The main body 511 may be constructed of the same aforementioned suitable materials. The side portion 513 may be constructed of an elastic, stretchable material or fabric. On an inner surface 506 of the main body 511, an adhesion member 526 is provided and situated between at least two projections 546 defining an elevated bridge portion 508.

In use, the device 500 is positioned on a limb, for example, a forearm (FIG. 12), a thigh or a foot (FIG. 13), with the exposed adhesion member 526 and projections 546 facing the skin above the treatment site. The side portions 513 are wrapped around the limb and releasably fastened to each other by fasteners 517, e.g., hook and loop fasteners, eye and hook fasteners, snap fasteners, etc. The device 500 in a neutral configuration presents the bridge portion 508 at an elevated position above the skin, the two separated by an air gap. When pressure is applied to outer surface 504 of the bridge portion 508, the device assumes a deformed configuration so that the adhesion member comes 526 into contact with the skin. When the pressure is removed, the device rebounds and the skin is lifted by the bridge portion. The device 500 may have a slight predetermined curvature or concavity in the L axis toward the inner surface 506 to better conform to the limb.

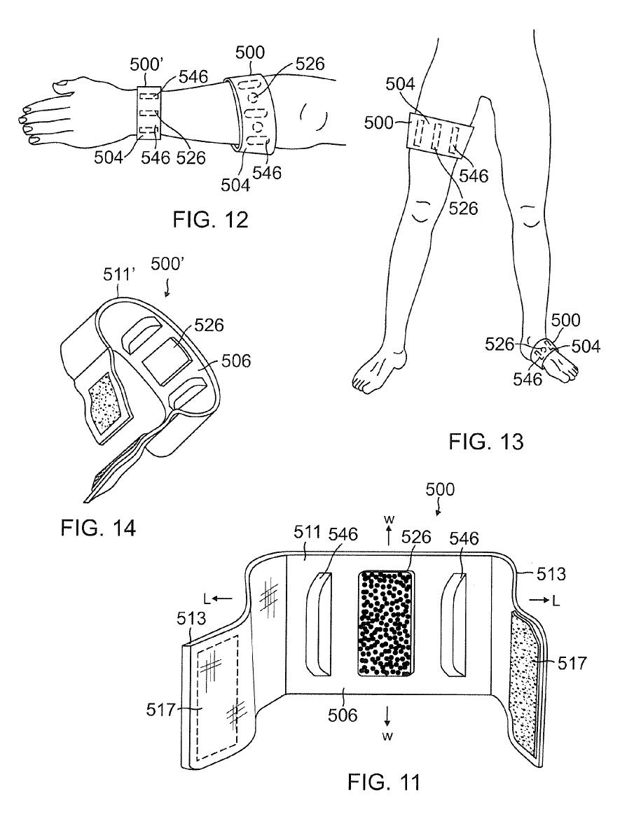

In a further embodiment of the present invention, a device 500′ may be worn as a cuff on the wrist, as shown in FIG. 12. The device 500′, as illustrated in FIG. 11, has a construction similar to that of the device 500 of FIG. 10, except that the device is smaller and has a pronounced predetermined curvature or concavity in the L axis toward the inner surface 506 to better conform to the wrist.

The present invention also includes a method of manufacturing a custom-fitted therapeutic device, wherein a mold of the treatment site, e.g., a palm, a forearm, a thigh, is made and a die set for manufacturing the device is customized according to the mold.

The preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention. As understood by one of ordinary skill in the art, the drawings are not necessarily to scale. Moreover, any one or more features of any embodiment may be incorporated in addition to or in lieu of any other one or more features of another embodiment, as appropriate or desired. Dimensions of the devices may be modified to fit different sized users. As noted above, the device may have more than one adhesive or adhesive member which may be applied to different areas of the device beyond the bridge portion. Accordingly, the foregoing description should not be read as pertaining only to the precise structures described and illustrated in the accompanying drawings, but rather should be read consistent with and as support to the following claims which are to have their fullest and fair scope.

What is claimed is:

1. A therapeutic device, comprising:
an elastically deformable sheet body having an elevated dome and a support portion, the elevated dome having an inner surface; and
an adhesive on the inner surface,
wherein the support portion has a base configured to contact a user's skin and the dome is configured to deform after adherence of the adhesive to a portion of the skin.

2. The therapeutic device of claim 1, wherein the dome has a first elevation and is configured to deform to a second elevation after adherence of the adhesive to the portion of the skin.

3. The therapeutic device of claim 1, wherein the dome after deformation is configured to pull on the skin after adherence of the adhesive to the portion of the skin.

4. The therapeutic device of claim 1, wherein at least a portion of the dome is configured to deform into a dimple after adherence of the adhesive to the skin.

5. The therapeutic device of claim 1, wherein the support portion includes at least one leg.

6. The therapeutic device of claim 1, wherein the support portion includes an annular flange.

7. The therapeutic device of claim 1, further comprising a plurality of legs supporting the elevated dome.

8. The therapeutic device of claim 7, wherein the plurality of legs ranges between about two and five.

9. A therapeutic device, comprising:
an elastically deformable sheet body having a dome and a base, the dome having an inner surface; and
an adhesive on the inner surface,
wherein the dome is configured to deform into a dimple around the adhesive.

10. The therapeutic device of claim 9, wherein the base has a generally circular shape.

11. The therapeutic device of claim 9, wherein the adhesive has a generally circular shape.

12. The therapeutic device of claim 9, wherein the base has a diameter ranging between about 1.5 inches and 10 inches.

13. A therapeutic device, having:
an elastically deformable body, the body having an elevated portion and a support portion, the body having an inner surface defining an inner volume of air;
an adhesive on the inner surface,
wherein the body is adapted to assume a deformed configuration with a vacuum in the inner volume of air when subjected to an external force depressing the elevated portion.

14. The therapeutic device of claim 13, wherein the body has a generally shallow frusto-conical configuration.

15. The therapeutic device of claim 13, wherein the support portion has a skirt configuration.

16. The therapeutic device of claim 13, wherein the elevated portion having a greater thickness and the support portion having a lesser thickness.
